(19) United States
(12) Patent Application Publication
(10) Pub. No.: US 2023/0355498 A1
(43) Pub. Date: Nov. 9, 2023

(54) HAIR GROWTH AGENT

(71) Applicants: ADJUVANT HOLDINGS CO., LTD., Kobe-shi, Hyogo (JP); RIKEN, Wako-shi, Saitama (JP)

(72) Inventors: Sota Nakamura, Kobe-shi (JP); Hideki Takahashi, Kobe-shi (JP); Yukimi Nakaike, Kobe-shi (JP); Takashi Tsuji, Wako-shi, Saitama (JP); Miho Ogawa, Kobe-shi (JP)

(73) Assignees: ADJUVANT HOLDINGS CO., LTD., Kobe-shi, Hyogo (JP); RIKEN, Wako-shi, Saitama (JP)

(21) Appl. No.: 18/028,386

(22) PCT Filed: Sep. 24, 2021
(86) PCT No.: PCT/JP2021/035018

§ 371 Date: Mar. 24, 2023

(30) Foreign Application Priority Data
Sep. 24, 2020 (JP) 2020-159791

(51) Int. Cl. A61K 8/64; A61Q 7/00

(52) U.S. Cl. CPC A61K 8/64; A61Q 7/00

(57) ABSTRACT

To provide an agent that increases expression of genes contributing to hair growth in dermal papilla cells, a scalp care agent, and a hair growth agent which are topical agents that exhibit effect in terms of causing increase in hair shaft diameter, and improving maximum hair shaft length, and improving hair shaft elongation rate, and new hair growth and increasing expression of genes contributing to hair growth in dermal papilla cells, and promoting hair shaft growth at head hair, beard, eyelashes, and/or eyebrows, such agents are made to contain an active ingredient in the form of palmitoyl dipeptide-5 diaminohydroxybutyrate.

### TECHNICAL FIELD

The present invention relates to a hair growth agent. More particularly, it relates to a hair growth agent that is a topical agent which contains palmitoyl dipeptide-5 diaminohydroxybutyrate.

### BACKGROUND ART

There has been increasing demand for hair growth agents and other such topical agents that will improve hair type and/or hair quality and hair growth effect in mammals including humans. To improve hair type and/or hair quality and hair growth effect, active ingredients which contribute to regulation of the hair cycle, i.e., the hair life cycle, have been proposed and are in the process of coming onto the market in the form of hair growth agents.

For example, use of minoxidil as an active ingredient in a hair growth agent has been proposed (see Patent Reference Nos. 1 through 3 and so forth), and hair growth agents employing minoxidil as active ingredient have undergone clinical trials in humans and are on the market. However, for reasons such as the fact that the pharmaceutical use thereof within Japan is limited to male alopecia prematura, it has not adequately satisfied the broad needs of consumers who desire hair growth effect and hair type and/or hair quality improvement effect.

Furthermore, use of chiro-inositol as active ingredient in hair growth agent has been proposed (see Patent Reference No. 4). However, as the hair growth effect of the hair growth agent which is a topical agent and which contains chiro-inositol that is described at Patent Reference No. 4 has only been demonstrated for non-insulin-resistant subjects, the subjects to whom it may be administered are limited. This being the case, it has not adequately satisfied the broad needs of consumers who desire hair growth effect and hair type and/or hair quality improvement effect.

Palmitoyl dipeptide-5 diaminohydroxybutyrate is known as a raw material for cosmetics (see Patent Reference No. 5). However, there are no reports related to a hair growth effect of palmitoyl dipeptide-5 diaminohydroxybutyrate.

### PRIOR ART REFERENCES

### Patent References

Patent Reference No. 1: Specification of U.S. Pat. No. 4,139,619
Patent Reference No. 2: Japanese Patent Application Publication Kokai No. S63[1988]-150211
Patent Reference No. 3: Japanese Patent Application Publication Kokai No. S63[1988]-145217
Patent Reference No. 4: International Patent Application Publication No. 2017/188393
Patent Reference No. 5: Japanese Patent No. 5028474

### SUMMARY OF INVENTION

### Problem to be Solved by Invention

It is an object of the present invention to provide a hair growth agent that possesses excellent hair growth action.

### Means for Solving Problem

As a result of intensive and repeated research for the purpose of solving the foregoing problems, the present inventor(s) discovered that use of palmitoyl dipeptide-5 diaminohydroxybutyrate as active ingredient permitted attainment of excellent hair growth activity, attainment of scalp care effect, and attainment of effect in terms of promoting dermal papilla cell FGF-7 production, which culminated in the present invention.

A first means in accordance with the present invention for solving the foregoing problems is a hair growth agent which is a topical agent that contains palmitoyl dipeptide-5 diaminohydroxybutyrate.

A second means in accordance with the present invention for solving the foregoing problems is the hair growth agent of the first means in accordance with the present invention wherein the palmitoyl dipeptide-5 diaminohydroxybutyrate is present therein in an amount that is 0.001 wt % to 20 wt % of the entirety.

A third means in accordance with the present invention for solving the foregoing problems is the hair growth agent of the first means or the second means in accordance with the present invention wherein the palmitoyl dipeptide-5 diaminohydroxybutyrate is present therein in an amount that is 0.005 wt % to 10 wt % of the entirety.

A fourth means in accordance with the present invention for solving the foregoing problems is the hair growth agent of any one among the first through third means in accordance with the present invention for use in causing new hair growth or hair shaft growth promotion.

A fifth means in accordance with the present invention for solving the foregoing problems is the hair growth agent of any one among the first through fourth means in accordance with the present invention used for causing improvement in hair shaft elongation rate.

A sixth means in accordance with the present invention for solving the foregoing problems is the hair growth agent of any one among the first through fourth means in accordance with the present invention used for causing improvement in maximum hair shaft length.

A seventh means in accordance with the present invention for solving the foregoing problems is the hair growth agent of any one among the first through fourth means in accordance with the present invention used for causing increase in hair shaft diameter.

An eighth means in accordance with the present invention for solving the foregoing problems is the hair growth agent of any one among the first through fourth means in accordance with the present invention used for causing increase in number of hairs.

A ninth means in accordance with the present invention for solving the foregoing problems is the hair growth agent of any one among the first through eighth means in accordance with the present invention in liquid solution form.

A tenth means in accordance with the present invention for solving the foregoing problems is the hair growth agent of any one among the first through ninth means in accordance with the present invention for use on head hair, beard eyelashes, and/or eyebrows.

An eleventh means in accordance with the present invention for solving the foregoing problems is a hair growth method comprising administering the hair growth agent of any one among the first through tenth means in accordance with the present invention to a subject.

Another means in accordance with the present invention for solving the foregoing problems is a scalp care agent which is a topical agent that contains palmitoyl dipeptide-5 diaminohydroxybutyrate.

Another means in accordance with the present invention for solving the foregoing problems is a scalp symptom improvement method comprising administering a scalp care agent which is a topical agent that contains palmitoyl dipeptide-5 diaminohydroxybutyrate to a subject.

Another means in accordance with the present invention for solving the foregoing problems is an agent for promoting dermal papilla cell FGF-7 production that contains palmitoyl dipeptide-5 diaminohydroxybutyrate.

### BENEFIT OF INVENTION

By causing palmitoyl dipeptide-5 diaminohydroxybutyrate to be an active ingredient in a hair growth agent which is a topical agent, means in accordance with the present invention make it is possible to provide an excellent hair growth agent, scalp care agent, and agent for promoting dermal papilla cell FGF-7 production that exhibit scalp care effect as well as effect in terms of causing increase in hair shaft diameter and improving maximum hair shaft length and improving hair shaft elongation rate and hair shaft growth promotion at head hair, beard, eyelashes, and/or eyebrows.

### BRIEF DESCRIPTION OF DRAWINGS

FIG. 1 are photographs for determining the situation with respect to new hair growth at location where drug was applied in mice, and graph showing change in hair shaft length at location where drug was applied in mice, following application of 0.05% solution of palmitoyl dipeptide-5 diaminohydroxybutyrate. The vertical axis of the graph shows hair shaft length (mm). Note that “placebo applied” indicates data after the fashion of a reference at which non-drug-containing 60% aqueous ethanol solution was applied during the first hair cycle.

FIG. 2 shows results of measurement of hair shaft diameter following application of drug.

FIG. 3 shows dermal papilla cell growth promotion effect as a result of stimulation with palmitoyl dipeptide-5 diaminohydroxybutyrate in human dermal papilla cells.

FIG. 4 shows change in amount of expression of FGF-7 gene as a result of stimulation with palmitoyl dipeptide-5 diaminohydroxybutyrate in human dermal papilla cells.

### EMBODIMENTS FOR CARRYING OUT INVENTION

Embodiments for carrying out the present invention are described below. Note that the present invention is not limited to these examples alone, it being of course possible to make any number of changes thereto without departing from the gist of the present invention.

The active ingredient of a hair growth agent and a scalp care agent that are topical agents associated with the present invention comprises palmitoyl dipeptide-5 diaminohydroxybutyrate (Palm-Lys-Val-Dab-OH).

Concentration of palmitoyl dipeptide-5 diaminohydroxybutyrate constituting the active ingredient in a hair growth agent and scalp care agent in accordance with the present invention is 0.001 wt % to 20 wt % of the entirety of the entire hair growth agent and scalp care agent. More specifically, it is 0.005 wt % to 10 wt %.

While hair growth agents and scalp care agents in accordance with the present invention may be used in the form of pharmaceutical preparations of any of a wide variety of modes such as ointments, poultices, liniments, lotions, liquids for topical use, dusting powders, creams, gels, emulsions, hair tonics, hair sprays, microneedles, and so forth as cosmetics including cosmetics for the scalp and cosmetics for the eyelashes and/or eyebrows, beard, head hair, quasi-pharmaceutical agents, pharmaceutical agents, and so forth, there is no limitation with respect thereto.

Furthermore, to the extent that it does not interfere with the hair growth effect and scalp care effect of the present invention, additives and/or other such components, presence of which would ordinarily be permitted in cosmetics including cosmetics for the scalp and cosmetics for the eyelashes and/or eyebrows, head hair, quasi-pharmaceutical agents, pharmaceutical agents, and so forth, may be additionally blended therein. As such additives and/or other such components, while excipients, stabilizers, corrigents, vehicle, dispersants, diluents, anionic surface active agents, amphoteric surface active agents, nonionic surface active agents, cationic surface active agents, anionic polymers, nonionic polymers, ethylene oxide—propylene oxide block copolymer, alcohols, emulsifiers, percutaneous absorption promoters, pH adjustors, preservatives, colorants, lipids, mineral oils, and other such oily components, moisturizing agents, thickeners, polymers, film-forming agents, ultraviolet light absorbers, cell activators, moisturizing agents, inorganic salts, functional beads and capsules, silicones, metal chelating agents, antioxidants, antiseptic agents, fresheners, deodorants, pigments, dyes, fragrances, sugars, amino acids, vitamins, organic acids, organic amines, plant extracts, clay minerals, various polymers, and other such viscosity modifiers, and so forth may be cited as examples, there is no limitation with respect thereto.

Hair growth agents and scalp care agents in accordance with the present invention may contain known components having new hair growth effect, hair growth effect, hair tonic effect, and/or the like.

Administration dosage of active ingredients per dose of a hair growth agent and scalp care agent of a means in accordance with the present invention may be adjusted so as to cause effect(s) of the hair growth agent and scalp care agent in accordance with the present invention to be exhibited. In addition, such administration dosage might for example be 0.005 mg to 200 mg, mi_{g}ht more specifically be 0.05 mg to 100 mg, and might still more specifically be 0.5 mg to 10 mg.

So as to cause effect(s) of the hair growth agent and scalp care agent in accordance with the present invention to be exhibited, the number of administrations of a hair growth agent and scalp care agent in accordance with the present invention might be one administration or might be multiple administrations. In addition, the number of administrations of a hair growth agent and scalp care agent in accordance with the present invention might for example be 1 to 6 times per day. In addition, more specifically this might be 1 to 3 times per day, and still more specifically this might be 1 to 2 times per day.

Hair growth agents and scalp care agents in accordance with the present invention relate to hair shaft growth promotion, new hair growth, and hair loss prevention, and preferably relate to hair shaft growth promotion and new hair growth.

In the present specification, the term “hair shaft growth promotion” means improving hair shaft elongation rate, improving maximum hair shaft length, and/or increasing hair shaft diameter.

In the present specification, the term “new hair growth” means promoting growth of new hair and increasing number of hairs at follicle pores where new hair growth capability has been lowered or where new hair growth has stopped at a location where there is a small number of hairs or where there is no hair (no hair shaft extends to the exterior from the epidermis), and more specifically means shortening the telogen phase of the hair cycle and/or restarting a stopped hair cycle.

In the present specification, “to have hair shaft growth promotion effect” means acting in a way such as will be advantageous for promotion of hair shaft growth, and the quality by which hair shaft growth promotion effect is indicated is referred to as “hair shaft growth promotion activity”. Furthermore, “to have new hair growth effect” means acting in a way such as will be advantageous for new hair growth, and the quality by which new hair growth effect is indicated is referred to as “new hair growth promotion activity”.

In the present specification, the term “hair loss” means the phenomenon whereby the hair shaft comes free from the follicle pore, and more specifically means increase in inhibitory cytokines or the like which interfere with cell growth, and to cell death resulting therefrom. The quality by which hair loss prevention effect is indicated is referred to as “hair loss prevention activity”. Furthermore, “to have hair loss prevention effect,” which is a physiological phenomenon different from the qualities by which hair shaft growth promotion and/or new hair growth effect are indicated, means decreasing the number of hair shafts that come free from follicle pores as a result of reduction in or interference with inhibitory cytokines and suppression of cell death.

In the present specification, the term “scalp symptoms” means dandruff, roughness of the scalp, dryness of the scalp, erythema, itchiness, acne, and/or other such symptoms. In addition, in the present specification, the term “improvement of scalp symptoms” means improvement or suppression of dandruff, roughness of the scalp, dryness of the scalp, erythema, itchiness, acne, and/or the like.

A hair growth agent in accordance with the present invention may be used to improve hair shaft elongation rate and/or maximum hair shaft length. In addition, with respect to hair shaft elongation rate, as compared with hair shaft elongation rate pursuant to hair cycle reference data, it may for example cause a maximum improvement of on the order of 110%, more specifically it may cause improvement on the order of 25% to 110%, and still more specifically it may cause improvement on the order of 33% to 110%. Furthermore, with respect to maximum hair shaft length, as compared with maximum hair shaft length pursuant to hair cycle reference data, it may for example cause a maximum improvement of on the order of 49%, more specifically it may cause improvement on the order of 1% to 49%, and still more specifically it may cause improvement on the order of 2% to 49%.

A hair growth agent in accordance with the present invention may be used to increase hair shaft diameter.

A hair growth agent in accordance with the present invention may be used to promote growth of new hair and increase the number of hairs at follicle pores where new hair growth capability has been lowered or where new hair growth has stopped at a location where there is a small number of hairs or where there is no hair (no hair shaft extends to the exterior from the epidermis), and more specifically may be used to shorten the telogen phase of the hair cycle and/or restart a stopped hair cycle.

Hair growth agents and scalp care agents in accordance with the present invention may be used not only for humans but also for animal pets and/or domesticated animals other than humans and/or other such animals (nonhuman animals). One aspect of the present invention provides a scalp symptom improvement method and a hair growth method that includes administration of a topical agent which contains palmitoyl dipeptide-5 diaminohydroxybutyrate to subject(s) which may include human(s), domesticated animal(s), animal pet(s), and/or other such animal(s).

### WORKING EXAMPLES

### Exemplary Test 1: Evaluation of Hair Growth Activity Caused by Mixture of Palmitoyl Dipeptide-5 Diaminohydroxybutyrate

### 1. Materials and Methods

### (1) Experimental Animals

C57BL/6N mice (male) and Balb/c nu/nu mice (female) were purchased from Japan SLC, Inc.(Japan) and bred, and were thereafter made available for the following testing. Note that the testing and breeding of animals complied with pertinent laws, regulations, ordinances, and guidelines, and was performed with the approval of the Experimental Ethics Review Board of the Institute of Physical and Chemical Research.

### (2) Reagents

The following reagents were respectively prepared.
Placebo: 60% aqueous ethanol solution
Working Example 1: 0.05% solution of palmitoyl
dipeptide-5 diaminohydroxybutyrate
   (3) Preparation of Skin Samples Derived from Mouse Dorsal Body Hair Skin

To collect dorsal body hair skin in the form of anagen stage VI skin 12 to 14 days following depilation, C57BL/6N mice of age 7 to 8 weeks were depilated at locations where dorsal body hair skin was intended to be collected, and were bred for 12 to 14 days. The depilated C57BL/6N mice were thereafter euthanized by cervical dislocation, following which a suitable amount of dorsal body hair skin was collected from the locations at which dorsal body hair skin was intended to be collected.

The collected skin was immersed in DMEM culture medium (hereinafter “DMEM 10”) which contained 10 mM HEPES, 10% fetal bovine serum, and 1% penicillin/streptomycin solution. The collected dorsal body hair skin was grasped with bent-nose curved tweezers and was treated by immersion for 10 seconds in a sterilizing solution. Sterilization treatment was performed by carrying out treatment with 7% povidone iodine solution two times, treatment with PBS (-) three times, and treatment with DMEM 10 two times, in this order, with fresh solutions respectively being used each time. Following sterilization treatment, these were immersed in clean DMEM 10.

Following sterilization treatment, the dorsal body hair skin was cut into pieces and formed into blocks. The transparent connective tissue which adhered to the cutaneous muscle layer of the skin was excised therefrom using curved scissors, and hair groups were cut into rectangular strips in parallel fashion with respect to the direction of the wave of the hair. At this time, these were cut into blocks such that there were 6 rows of hair follicles along the long axis, adjustment having been carried out so that there were 5 rows of hair follicles along the short axis.

(4) Grafting of Skin Samples onto Balb/c nu/nu Mice

The skin samples derived from dorsal body hair skin that were prepared in accordance with the foregoing were grafted onto Balb/c nu/nu mice of age 4 to 6 weeks.

More specifically, mice were anesthetized in the usual way using isoflurane gas. The dorsal area of the mice was then disinfected using 7% povidone iodine solution, following which the mice were made to assume a naturally recumbent posture. In addition, a Mani ophthalmic knife (Mani, Inc.; Japan) was used to pierce the skin at the dorsal area of the mice, the grafts which were formed extending from the epidermal layer of the skin to the subdermal layer. The skin samples derived from dorsal body hair skin were inserted into the grafts formed thereat in such fashion as to cause the hair groups to be directed toward the body surface side of the grafts. Skin sample transplanted depth was adjusted so as to cause the top portion of the hair group to be in a state such that it was exposed at the top portion of the graft. To protect the grafts, Nurseban (registered trademark) (Sunplanet Co., Ltd.; Japan) and surgical tape (3M Japan Limited; Japan) were then used as protective tape to cover the grafts at which the skin samples derived from dorsal body hair skin had been transplanted. The protective tape was removed 5 to 7 days following transplantation, and survival of the transplanted skin samples derived from dorsal body hair skin was determined by visual inspection or digital microscopy (Keyence Corporation: Japan), after which follow-up observation was carried out.

### (5) Application of Drug on Transplanted Skin Samples

For the first hair cycle, 60% aqueous ethanol solution was applied thereto as placebo. A micropipette was used to apply 25 μL of 60% ethanol respectively to the left and right dorsal regions of skin samples that survived in Balb/c nu/nu mice in which skin samples had been transplanted in accordance with the foregoing. A dryer was thereafter used to cause cool air to be directed thereat and rapidly dry the ethanol. This procedure was carried out in repetitive fashion four times at each the left and right dorsal regions of the mice.

For the second and subsequent hair cycles, palmitoyl dipeptide-5 diaminohydroxybutyrate solution was applied instead of 60% aqueous ethanol solution in accordance with the foregoing method to the Balb/c nu/nu mice in which the hair groups had been transplanted.

### (6) Histologic Analysis and Follow-Up Observation of New Hair Growth

Three regions were selected from the locations at which the skin samples were transplanted in Balb/c nu/nu mice, the situation with respect to new hair growth being determined and recorded for five hairs selected from each of these regions. Observation and recording was carried out by visual inspection and digital microscopy (Keyence Corporation; Japan).

### 2. Results

For each drug, hair shaft length was measured once every 2 to 4 days, the average of the hair shaft lengths at any given time being plotted as a single data point on a graph showing the change thereof with respect to time, similar plots being made for each of three mice. Results are shown in TABLE 1 and in FIG. 1.

TABLE 1

| | | Data for situation in which |
|---|---|---|
| | Placebo applied | drug acted thereon |
| | (first hair cycle) | (second hair cycle) |
| Hair shaft elongation | 0.38 ± 0.06 | 0.39 ± 0.06 |
| rate (mm/day) | | |
| Percent change relative | | 100.63% |
| to reference data | | |
| Maximum hair shaft | 4.9 ± 0.4 | 5.2 ± 0.2 * |
| length (mm) | | |
| Percent change relative | | 105.19% |
| to reference data | | |

Here, * at TABLE 1 and FIG. 1 indicates p<0.05, i.e., that the results are significant.

Wien a solution containing 0.05% of palmitoyl dipeptide-5 diaminohydroxybutyrate was applied to the locations at which the skin samples had been transplanted in mice, there was improvement in hair shaft elongation rate as compared with reference data. Furthermore, with respect to maximum hair shaft length, there was significant improvement (see TABLE 1 and FIG. 1). Based on these results, the solution containing 0.05% of palmitoyl dipeptide-5 diaminohydroxybutyrate was found to exhibit hair growth activity.

### Exemplary Test 2: Evaluation of Hair-Diameter-Increasing Activity Caused by Palmitoyl Dipeptide-5 Diaminohydroxybutyrate

### 1. Materials and Methods

### (1) Method for Measuring Increase in Hair Diameter

Measurement of increase in hair diameter was carried out using hair shafts that had completed the second cycle at the foregoing Exemplary Test 1. Of the hair shafts that were collected, three zigzag hairs were used for measurement of increase in hair diameter. Three locations were selected in regions where diameter was large in the central portions thereof using a square 100 μm on a side. For each of the selected regions, five different locations were further selected, and hair shaft diameter of the zigzag hairs thereat was measured to evaluate the degree to which increase in hair diameter had occurred.

### 2. Results

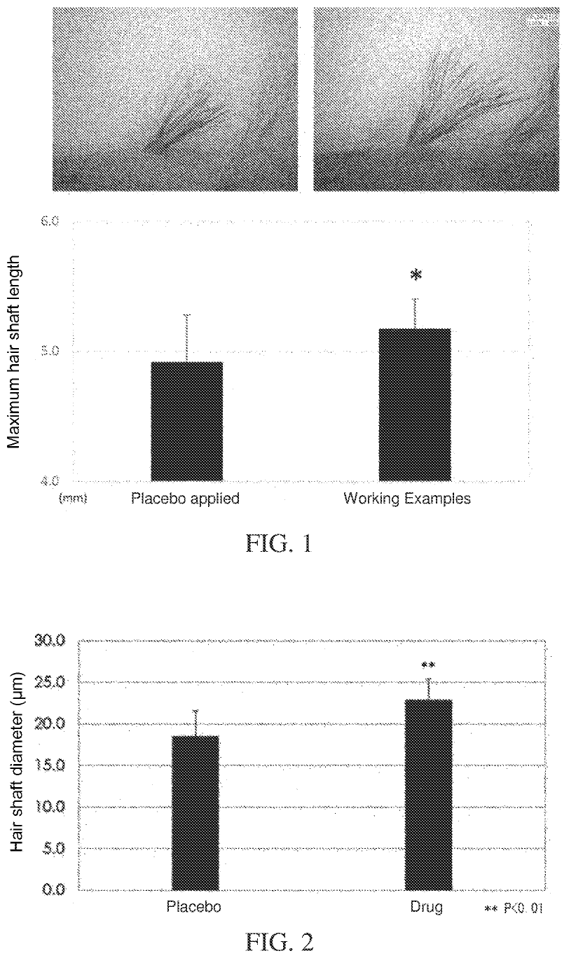

Results of measurement of hair shaft diameter are shown in FIG. 2 and in TABLE 2. Here, * at FIG. 2 and TABLE 2 indicates p<0.01, i.e., that the results are significant.

TABLE 2

| | | Data for situation in |
|---|---|---|
| | Placebo applied | which drug acted thereon |
| | (first hair cycle) | (second hair cycle) |
| Hair shaft diameter (μm) | 18.5 ± 3.1 | 22.9 ± 2.5 ** |
| Percent increase (%) | | 124 |

** p < 0.01

It was determined that application of palmitoyl dipeptide-5 diaminohydroxybutyrate, which is the active ingredient of a means in accordance with the present invention, caused occurrence of a clear increase in hair diameter as compared with the diameters of hair shafts which had completed the second cycle and on which 60% aqueous ethanol solution serving as control had been applied.

### Exemplary Test 3: Evaluation of Hair-Diameter-Increasing Activity Caused by Palmitoyl Dipeptide-5 Diaminohydroxybutyrate

### 1. Materials and Methods

### (1) Human Dermal Papilla Cells and Culture Medium

Human dermal papilla cells (Catalog No. CA602t05a; Caucasian; derived from 29-year-old male; Toyobo Co., Ltd. (Japan)) were purchased, testing and evaluation being carried out with maintenance and culture of cells being performed as described in the protocol.

### (2) Drugs

As drugs for testing, drug solutions consisting of culture medium for human dermal papilla cells containing palmitoyl dipeptide-5 diaminohydroxybutyrate in the following respective concentrations (final concentrations) were prepared, and these were used.
Working Example 1: 11.54709375 μM
Working Example 2: 23.0941875 μM
Working Example 3: 46.188375 μM
Working Example 4: 92.37675 μM
Working Example 5: 184.7535 μM
Working Example 6: 369.507 μM

### (3) Test Procedure

A 96-well plate was seeded with human dermal papilla cells so as to obtain 1×10³ thereof per well. Following culture for 1 day within a CO₂ incubator (5% CO₂; 37° C.), human dermal papilla cell culture medium was replaced with drug solutions consisting of culture medium for human dermal papilla cells containing palmitoyl dipeptide-5 diaminohydroxybutyrate in the foregoing respective concentrations. The cell plate was thereafter returned to the CO₂ incubator, and this was further cultured for 24 hours, 48 hours, or 72 hours. Following culture, the culture supernatant was discarded, and the cells were washed with phosphate buffer physiological saline solution (abbreviated as “PBS”). Following washing with PBS, 100 μL/well of culture medium containing 10% of Cell Count Reagent SF (Nacalai Tesque, Inc. (Japan)) was added thereto. After this had been added thereto, absorbance (measurement wavelength 450 nm; reference wavelength 620 nm) of the culture supernatant was measured. Based on these values, the cell growth rates at the respective wells were calculated, at which time the cell growth rate of the control group at which nothing had been added was taken to be 100%.

### 2. Results

The change in viable cell rate as a function of time following action of palmitoyl dipeptide-5 diaminohydroxybutyrate on human dermal papilla cells was measured, the results thereof being shown in FIG. 3.

As shown in FIG. 3, it was found that causing palmitoyl dipeptide-5 diaminohydroxybutyrate to act on human dermal papilla cells (Working Example 1 through Working Example 6) resulted in an increase in cell growth rate as compared with the control group at which nothing had been added. Moreover, within the concentration domain tested in the present study, it was found that palmitoyl dipeptide-5 diaminohydroxybutyrate induced cell growth in concentration-dependent fashion, the peak of which occurred for the concentration at Working Example 5.

### Exemplary Test 4: Evaluation of FGF-7 Gene Expression in Human Dermal Papilla Cells as a Result of Palmitoyl Dipeptide-5 Diaminohydroxybutyrate

### 1. Materials and Methods

### (1) Human Dermal Papilla Cells and Culture Medium

Testing and evaluation were carried out with maintenance and culture of human dermal papilla cells being performed in the same manner as at Exemplary Test 3, above.

### (2) Drugs

As drugs for testing, drug solutions of the following respective concentrations (final concentrations) were prepared and used.
Comparative Example 1: 100 μM adenosine
Working Example 1: 1 μM palmitoyl dipeptide-5 diaminohydroxybutyrate
Working Example 2: 10 μM palmitoyl dipeptide-5 diaminohydroxybutyrate
Working Example 3: 300 μM palmitoyl dipeptide-5 diaminohydroxybutyrate

### (3) Test Procedure

A 24-well plate was seeded with human dermal papilla cells so as to obtain 6×10³ thereof per well. Following culture for 1 day within a CO₂ incubator (5% CO₂; 37° C.), the culture medium was replaced with culture medium which contained the respective drugs for testing. The cell plate was thereafter returned to the CO₂ incubator, and this was further cultured for 24 hours.

Following culture, total RNA was extracted from the respective wells and was recovered, and this was reverse-transcribed into cDNA. The cDNA that was prepared was used to measure FGF-7 gene expression in accordance with the real-time PCR method. The GAPDH gene was used as an internal standard, the amount of FGF-7 gene expression being calculated relative to the negative control group.

A FastGene RNA Basic Kit (Catalog No. FG-80250; Nippon Genetics Co., Ltd. (Japan)) was used to recover total RNA from cells. 300 μL of lysis buffer RL was added thereto per well, and the cells were lysed by pipetting. 300 μL of 70% ethanol was added to the cell lysate, and this was mixed by pipetting. The sample solution was added to a FastGene RNA binding column, and this was centrifuged at room temperature for 1 minute at 10000 g. The filtrate that passed through the column was discarded from the collection tube, and after returning the FastGene RNA binding column to its original collection tube, 600 μL of wash buffer RW1 was added to the FastGene RNA binding column, and this was centrifuged at room temperature for 1 minute at 10000 g. The FastGene RNA binding column was transferred to a new collection tube that was placed thereat, 700 μL of wash buffer RW2 was added to the FastGene RNA binding column, and this was centrifuged at room temperature for 1 minute at 10000 g. The FastGene RNA binding column was transferred to a new collection tube that was placed thereat, and this was centrifuged at room temperature for 1 minute at 15000 g. The FastGene RNA binding column was transferred to a new collection tube that was placed thereat, 50 μL of elution buffer RE was added at the center of the membrane of the FastGene RNA binding column, and this was centrifuged at room temperature for 1 minute at 10000 g to recover the purified RNA. Concentration of the recovered RNA was measured using a NanoDrop Lite (Catalog No. ND-LITE; Thermo Fisher Scientific K.K.), and this was stored at −80° C. until the following cDNA creation procedure.

A FastGene scriptase II cDNA synthesis 5× Ready Mix (Catalog No. NE-LS641; Nippon Genetics Co., Ltd. (Japan)) was used to synthesize cDNA. Dilution with RNase-free Water was carried out so as to cause concentration of total RNA produced in a new tube to be 20 ng/mL, 4 μL of FastGene scriptase II cDNA synthesis 5× Ready Mix was added to 16 μL of this sample solution, and this was agitated by vortexing. A MiniAmp thermal cycler (Thermo Fisher Scientific K.K.) was used to incubate this at 25° C. for 10 minutes, 42° C. for 60 minutes, and 85° C. for 5 minutes to synthesize cDNA.

The cDNA that was synthesized in accordance with the foregoing method was used to carry out real-time PCR. At prescribed wells in a 96-well plate, respective dilute solutions of cDNA template were added, Thunderbird SYBR qPCR Mix (Catalog No. QPS-201; Toyobo Co., Ltd. (Japan)) and primer were added thereto and mixed therewith, and gene expression was analyzed using a QuantStudio 7 Flex Real-Time PCR System (Catalog No. 4485693; Thermo Fisher Scientific k.K ). The PCR reaction was such that 40 cycles of 95° C. for 5 seconds, and 60° C. for 30 seconds, were carried out.

Primers specific for the FGF-7 gene, and primers specific for the GAPDH gene which was used as internal standard, these having been used for testing, are indicated below.
Primers for detecting FGF-7 gene expression
   Forward: gagagaaaatccttctgcctgttg (Sequence No. 1)
   Reverse: cctggtgcaacttgagcctt (Sequence No. 2)
Primers for detecting GAPDH gene expression
   Forward: catccctgcctctactggcgctgcc (Sequence No. 3)
   Reverse: ccaggatgcccttgagggggccctc (Sequence No. 4)

Relative amounts of expression of the respective genes were calculated as follows.

For each gene, Ct value (number of PCR cycles) was calculated based on the intersection of the amplification curve with the threshold line. The relative amount of expression is the target gene Ct value less the internal standard GAPDH gene Ct value.

### 2. Results

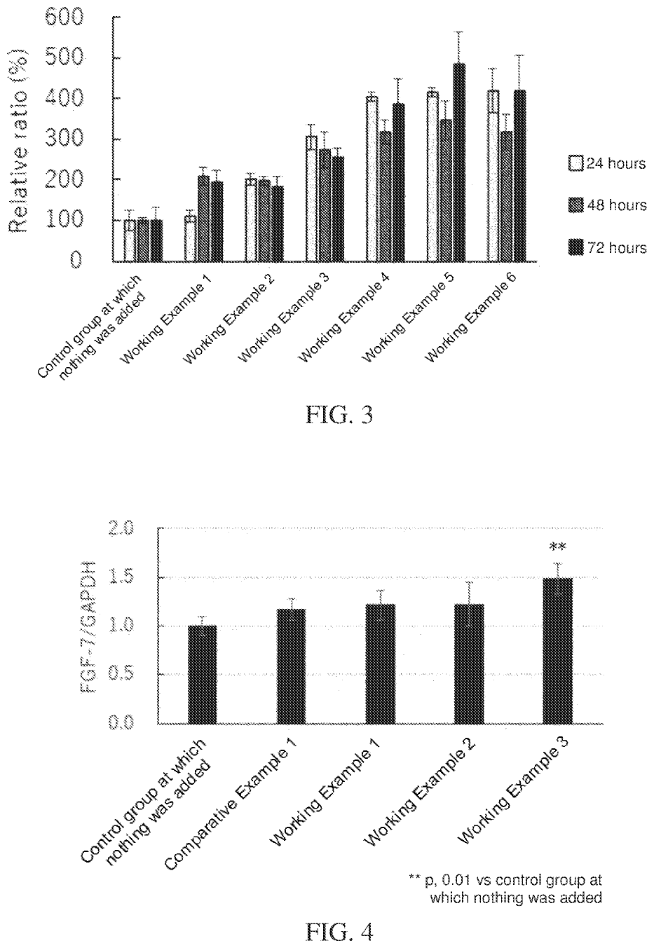

The change in the amount of expression of the FGF-7 gene after palmitoyl dipeptide-5 diaminohydroxybutyrate was allowed to act on human dermal papilla cells for 24 hours was measured, the results thereof being shown in FIG. 4. Here, ** at FIG. 4 indicates p<0.01, i.e., that the results are significant.

As shown in FIG. 4, it was found that causing palmitoyl dipeptide-5 diaminohydroxybutyrate to act for 24 hours on human dermal papilla cells (Working Examples 1 and 2) resulted in an increase in the amount of expression of the FGF-7 gene as compared with the control group at which nothing had been added. In addition, it was found that increasing the amount of palmitoyl dipeptide-5 diaminohydroxybutyrate that was added thereto caused the amount of expression of the FGF-7 gene to be greater than that which was produced as a result of action of adenosine (Comparative Example 1).

### INDUSTRIAL UTILITY

As a result of using palmitoyl dipeptide-5 diaminohydroxybutyrate as active ingredient in a hair growth agent which is a topical agent, a means in accordance with the present invention makes it possible to provide a novel scalp care agent and hair growth agent that exhibit promotion of dermal papilla cell FGF-7 production agent and scalp care effect, effect in terms of improving maximum hair shaft length, effect in terms of improving hair shaft elongation rate, and hair shaft growth promotion effect at head hair, beard, eyelashes and/or eyebrows, and/or other such hair.

1. A hair growth agent which is a topical agent that contains palmitoyl dipeptide-5 diaminohydroxybutyrate.

2. The hair growth agent according to claim 1 wherein the palmitoyl dipeptide-5 diaminohydroxybutyrate is present therein in an amount that is 0.001 wt % to 20 wt % of the entirety.

3. The hair growth agent according to claim 1 wherein the palmitoyl dipeptide-5 diaminohydroxybutyrate is present therein in an amount that is 0.005 wt % to 10 wt % of the entirety.

4. The hair growth agent according to claim 1 for use in causing new hair growth or hair shaft growth promotion.

5. The hair growth agent according to a claim 1 used for causing improvement in hair shaft elongation rate.

6. The hair growth agent according to claim 1 used for causing improvement in maximum hair shaft length.

7. The hair growth agent according to claim 1 used for causing increase in hair shaft diameter.

8. The hair growth agent according to a claim 1 used for causing increase in number of hairs.

9. The hair growth agent according to a claim 1 in liquid solution form.

10. The hair growth agent according to claim 1 for use on head hair, beard, eyelashes, and/or eyebrows.

11. A hair growth method comprising administering the hair growth agent according to claim 1 to a subject.
